(19) United States
(12) Patent Application Publication
(10) Pub. No.: US 2005/0016882 A1
(43) Pub. Date: Jan. 27, 2005

(54) Optometric trial lens set apparatus and method

(76) Inventors: Ravindra Kankaria, Houston, TX (US); Hongtao Lu, Houston, TX (US)

Correspondence Address: LUNDEEN & DICKINSON, LLP, PO BOX 131144, HOUSTON, TX, 77219-1144, US

(21) Appl. No.: 10/614,358

(22) Filed: Jul. 7, 2003

(51) Int. Cl. A61B 3/04; B65D 85/38

(52) U.S. Cl. 206/316.1

(57) ABSTRACT

An optometric trial lens set, a bifurcated case therefor, and method of use thereof are disclosed. Preferably, the case includes a first compartment, a second compartment, and a divider caddy therebetween. The first compartment is preferably configured to retain the trial lens set whereas the second compartment and the divider caddy are configured to retain a plurality of optometric instruments. Optionally, an electroluminance lamp index system is provided to enable an eye-care professional to identify components of the trial lens set in partial or complete darkness.

### BACKGROUND OF THE INVENTION

Doctors of Optometry and Ophthalmology and various other eye-care professionals frequently use advanced refraction devices, whether manual or automatic, in examining patients. Often, a preliminary refraction is accomplished by an auto-refractor with a fine-tuned adjustment to be performed on a manual refraction instrument. If necessary, additional fine-tuning may be accomplished through manual comparison testing with a trial lens set.

A trial lens set consists of a universal holder, or trial frame, and an array of precision ground reference lenses. The eye-care professional places the holder upon the patient and selectively places trial lenses within the holder until the patients eyesight is properly corrected. The eye-care professional then notes the power of the trial lenses in making an eye glasses prescription for the patient. Often, an eye-care professional will use a lens from a trial lens set to add or remove correction to existing corrective lenses to fine tune in smaller increments not possible with other equipment.

While the manual comparison testing represents the most rudimentary and antiquated method for defining a patient's prescription, it is a method that all eye-care physicians must learn and use. Additionally, when a patient is incapable of making an office visit, the eye-care professional must take his or her instruments to the patient. Because most auto and manual refractors are large, bulky devices, a trial lens set is often taken. Furthermore, trial lens sets are more economical than manual and auto refractors and every student of Optometry is required to carry their trial lens set to clinics. While trial lens sets are more portable than a refractor, no portable system for carrying a complete trial lens set and other optometric equipment exists. A need for a portable optometric system exists for eye-care professionals that must travel to their patients, including, but not limited to, doctors performing charity work in developing countries. Since every eye-care professional in training is required to have a trial lens set, a need arises for a easily transportable system to carry and use them along with some other rudimentary optometric instruments. Furthermore, because most eye-care professionals perform their examinations in the darkened environments, selecting a particular trial lens can be difficult. A need exists for a system to help an eye-care professional find and select trial lenses in the dark.

### SUMMARY OF THE INVENTION

Preferred embodiments of the present invention are disclosed and include an optometric trial lens set comprising a bifurcated case having a first compartment, a second compartment and a divider caddy therebetween. Preferably, the first compartment and second compartment are connected together with a hinge. The first compartment is configured to house an array of optometric trial lenses. The second compartment and a display surface of the divider caddy are configured to house a plurality of optometric instruments. The divider is configured to convert from a position that allows the use of the optometric trial lenses to a position that covers the optometric trial lenses but allows the use of a the optometric instruments. The preferred embodiment optionally includes an electroluminance system to provide for easy visual selection of trial lenses from the trial lens set in darkened environments.

### BRIEF DESCRIPTION OF THE DRAWINGS

For a detailed description of the preferred embodiments of the invention, reference will now be made to the accompanying drawings in which:

FIG. 1 is a perspective view drawing of an optometric trial lens set and case in accordance with a preferred embodiment of the present invention showing a divider caddy in a first position.

FIG. 2 is a perspective view drawing of the optometric trial lens set and case of FIG. 1 showing the divider caddy in a second position.

FIG. 3. is a perspective view drawing of the case of FIGS. 1-2 in a closed configuration showing a handle, shoulder strap, and a logo thereattached.

### DETAILED DESCRIPTION OF THE DRAWINGS

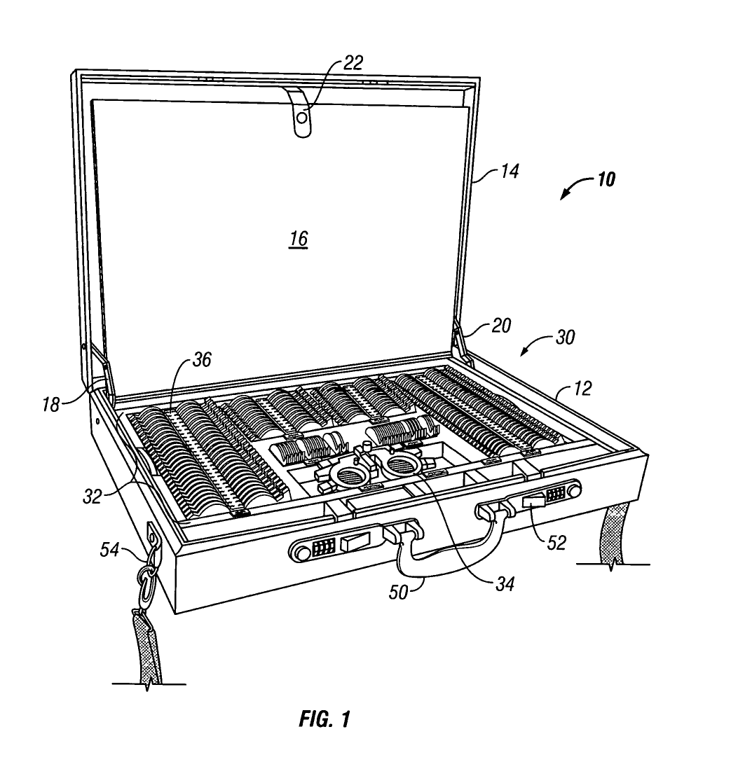

Referring initially to FIG. 1, a case 10 to house an optometric trial lens set 30 is shown. Case 10 is preferably of a bifurcated construction and includes a first compartment 12, a second compartment 14, and a divider caddy 16. First compartment 12 and second compartment 14 are preferably connected through a hidden piano hinge (not visible) but any hinge-like apparatus may be employed. Struts 18 and 20 provide rigidity between first compartments 12 and 14 and prevent second compartment 14 from being opened too far. Preferably, compartments 12 and 14 open at a 90 degree angle with first compartment 12 being horizontal and second compartment 14 being substantially (±15°) vertical in relation to each other. An alternative configuration (not shown) for lens set and case 10 may involve compartments 12 and 14 opening up substantially horizontally. FIG. 1 shows divider caddy 16 in a vertical configuration blocking access to second compartment 14 and being held in place by snap 22. In this configuration, access to contents of first compartment 12 is provided.

First compartment 12 preferably houses optometric trial lens set 30. Trial lens set 30 typically includes a plurality of precision ground trial lenses 32 and a universal holder apparatus, or trial frame 34. Trial lens set 30 is preferably employed by placing trial frame 34 upon a patient and selectively installing alternative configurations of trial lenses 32 in frame 34 until the desired correction is obtained. As can be appreciated, first compartment 12 includes an indexing and holding scheme to secure lenses 32 in an array that allows the eye-care professional to easily find, use, and replace a particular lens 32 with minimal effort. To accomplish this, first compartment 12 preferably contains a plurality of parallel scalloped grooves, each configured to hold lenses 32. Furthermore, compartment 12 is preferably coated in a material, either natural or synthetic, that is selected to minimize shock and scratches to the precision ground lenses 32. Examples of such materials include, but are not limited to, silk, velvet, plush cotton, or synthetic microfiber materials.

Next, adjacent to rows of trial lenses 32, lens indexing markers 36 are located. Indexing markers 36 can include stamped, printed, or etched characters to easily and effectively identify the particular attributes of each lens 32 of optometric trial lens set 30 closest thereto. In a preferred embodiment of the present invention, indexing markers 36 are constructed with electroluminance lamps that “glow” slightly when an electrical potential is applied thereto. The primary benefit of using electroluminance lamps for indexing markers 36 is that they allow the eye-care professional to quickly identify, select, and use trial lenses 32 from the case and set 10, 30 in complete darkness or diminished light. Because diminished light situations are preferable for most eye examinations, this feature would be of great benefit to any practicing eye-care professional.

Electroluminance indexing markers 36 can be constructed to either as a strip adhered to the top of the material of first compartment 12, between rows of lenses 32 or may be built-in to compartment 12. A “built-in” configurations would have the majority of markers 36, and any wires, electronics, or power supplies thereattached, hidden beneath the material of compartment 12. Preferably, in the built-in configuration, the strip of electroluminance material would lie below the material of first compartment 12 between two rows of lenses 32, with only an upset portion housing the actual identifying characters of markers 36 showing through the protective material of compartment 12. This arrangement minimizes clutter and allows accurate identification of each lens 32 of set 30. Furthermore, as electroluminance indexing markers 36 are preferably battery powered, a switch (not shown) could be incorporated into trial lens set 10 to maximize battery life. Potentially, a microswitch (not shown) could be incorporated into case 10 such that when top 14 and bottom 12 compartments are closed, the electroluminance markers 36 are switched off.

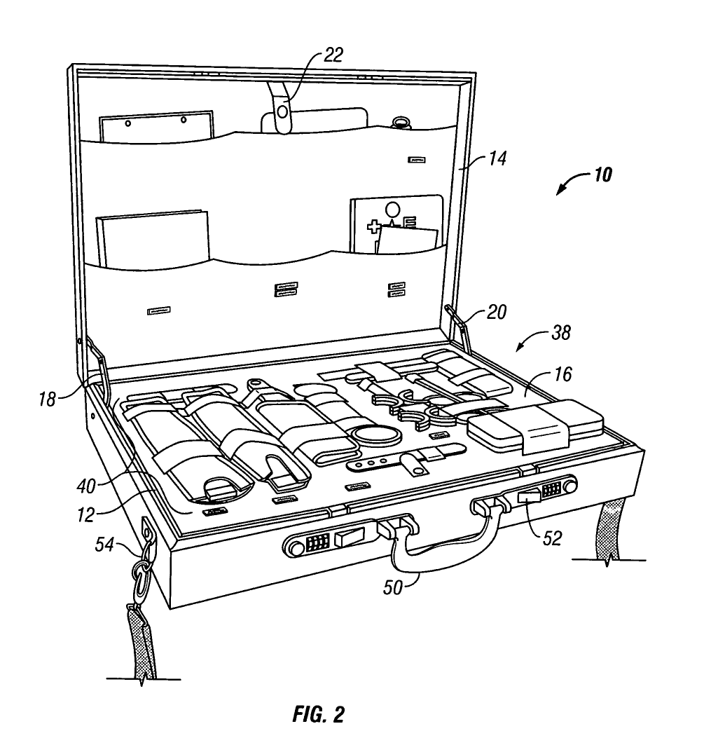

Referring now to FIG. 2, divider caddy 16 is shown in a horizontal configuration, a configuration that covers lens set 30 housed within first compartment 12, but exposes second compartment 14 and a tool holder side 38 of divider caddy 16. Preferably, second compartment 14 and tool holder side 38 of divider caddy 16 retain a plurality of optical instruments 40. Plurality of optical instruments includes, but is not limited to, any standard instruments that an eye-care professional might require during the course of an examination.

The exact contents of plurality of instruments 40 will change through the preferences of each trial lens set 10 owner. Specifically, optical instruments expected to be carried within second compartment 14 and upon divider caddy 16 include: prism bars, opthalmoscopes, retinoscopes, retinoscopy racks, Maddox occluders, near vision testing charts, well flippers, accommodative flippers, ocular foreign removal kits, color vision testing kits, stereo vision testing kits, lens clocks, contact lens reticules, PD rulers, Worth 4 dot testing kits, and blood pressure measuring kits. Furthermore, second compartment 14, shown in FIG. 2 configured to hold various forms of literature, charts, and examination guides, may instead be configured to retain patient files, or other documents the eye-care professional may deem necessary. Notwithstanding the categorization above, optical tools, accessories, literature, patient files, charts, guides and the like are all considered to be within the definition of “optical instruments.”

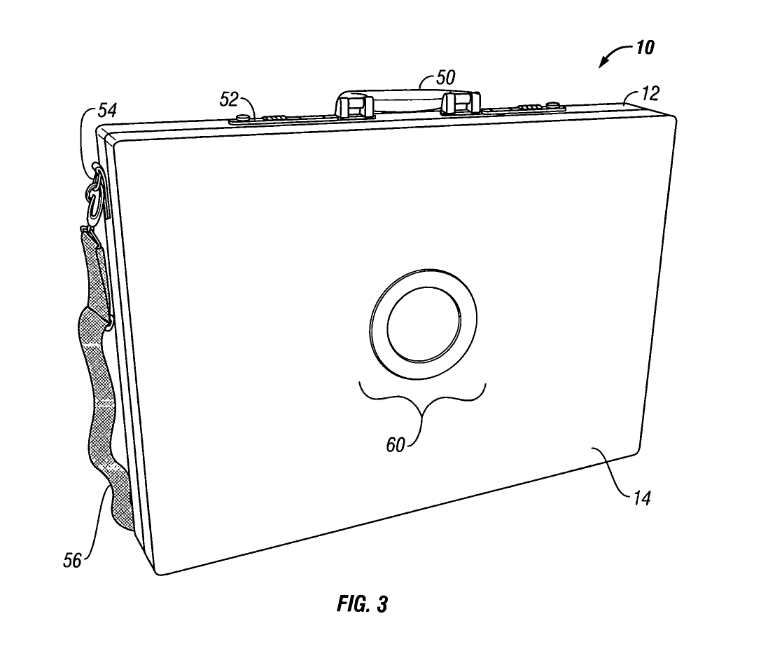

Referring finally to FIG. 3, the case 10 for trial lens set 30 is shown in a closed, carrying configuration. In the configuration of FIG. 3, first and second compartments 12, 14 are closed together. A handle 50, is provided to aid in lifting case 10 and trial lens set 30 and a locking mechanism 52 secures compartments 12, 14 in a closed position to prevent unauthorized access to their contents. Optionally, lugs 54 are provided on either end of case 10 so that a shoulder carrying strap 56 may be attached and used. Additionally, a logo 60 designating a brand name, professional affiliation, or an eye-care professional's identification may be embossed or otherwise affixed to the outside of either compartment 12, 14. Finally, wheels and a long handle (not shown) may be attached to the bottom of trial lens set 10 in a manner consistent carry-on luggage currently well known in the art to allow easy transportation where the surface traveled allows.

While a preferred embodiment of the invention has been shown and described, modifications thereof can be made by one skilled in the art without departing from the spirit of the invention.

1. An optometric trial lens set comprising:
a bifurcated case, said bifurcated case having a first compartment, a second compartment, and a divider caddy therebetween;
said first compartment hingedly connected to said second compartment;
said first compartment configured to display and retain an array of optometric trial lenses;
said second compartment configured to retain a first plurality of optometric instruments;
said divider caddy configured to hingedly convert from a first position to a second position;
said first position configured to expose and allow the use of said array of optometric trial lenses;
said second position configured to cover said array of optometric trial lenses, expose said first plurality of optometric instruments, and to expose a display surface of said divider caddy; and
said display surface configured to retain a second plurality of optometric instruments.

2. The optometric trial lens set of claim 1 wherein said first compartment provides a plurality of scalloped recesses to retain said optometric trial lenses.

3. The optometric trial lens set of claim 2 wherein said first compartment is lined with a non-abrasive material to cushion and protect said optometric trial lenses against scratches and shocks.

4. The optometric trial lens set of claim 1 wherein said first compartment is configured to be horizontal when said bifurcated case is in an open position.

5. The optometric trial lens set of claim 4 wherein said second compartment is configured to be substantially vertical when said bifurcated case is in said open position.

6. The optometric trial lens set of claim 5 wherein said first position of said divider caddy is parallel to said second compartment and said second position of said divider caddy is parallel to said first compartment.

7. The optometric trial lens set of claim 6 wherein said divider caddy is lockable into said first position.

8. The optometric trial lens set of claim 1 wherein said bifurcated case includes a carry handle.

9. The optometric trial lens set of claim 1 wherein said bifurcated case includes securing rings for a shoulder strap.

10. The optometric trial lens set of claim 1 wherein said bifurcated case includes at least one pair of wheels and a tow handle.

11. The optometric trial lens set of claim 1 wherein said first compartment is configured with electroluminance lamp indexing markers to identify lenses within the optometric trial lens set.

12. The optometric trial lens set of claim 11 further including a switch to activate and deactivate said indexing markers when said bifurcated case is opened and closed.

13. An case for an optometric trial lens set, the case comprising:
a first compartment, a second compartment, and a divider caddy therebetween;
said first compartment hingedly connected to said second compartment;
said first compartment configured to display and retain the optometric trial lens set;
said second compartment configured to retain a first group of optometric instruments;
said divider caddy configured to hingedly convert from a first position to a second position;
said first position configured to expose and allow the use of the optometric trial lens set;
said second position configured to cover the trial lens set, to expose said first group of optometric instruments, and to expose a display surface of said divider caddy; and
said display surface configured to retain a second group of optometric instruments.

14. The case of claim 13 wherein said first compartment provides a plurality of scalloped recesses to retain said optometric trial lenses.

15. The case of claim 13 wherein said first compartment is configured to be horizontal when the case is in an open position.

16. The case of claim 15 wherein said second compartment is configured to be substantially vertical when the case is in said open position.

17. The case of claim 16 wherein said first position of said divider caddy is parallel to said second compartment and said second position of said divider caddy is parallel to said first compartment.

18. The case of claim 13 further comprising a carry handle.

19. The case of claim 13 further comprising securing rings for a shoulder strap.

20. The case of claim 13 further comprising at least one pair of wheels and a tow handle.

21. The case of claim 13 further comprising a logo upon an outer surface of the case.

22. The case of claim 13 wherein said first compartment is configured with electroluminance lamp indexing markers to identify lenses within the optometric trial lens set.

23. The case of claim 22 further including a switch to activate and deactivate said indexing markers when the case is opened and closed.

24. A method to transport, store, and use an optometric trial lens set, the method comprising:
securing a plurality of trial lenses in a horizontal compartment of a bifurcated case, the bifurcated case also having a vertical compartment and a divider caddy therebetween;
securing a first group of optometric instruments within the vertical compartment;
securing a second group of optometric instruments upon a display surface of the divider caddy;
hingedly positioning the divider caddy between the horizontal and the vertical compartments of the bifurcated case;
accessing the first and second groups of optometric instruments when the divider caddy is in a horizontal position; and
accessing the plurality of trial lenses accessible when the divider caddy is in a vertical position.

25. The method of claim 24 further comprising installing electroluminance lamp indexing markers within the horizontal compartment of the bifurcated case adjacent to the plurality of trial lenses.

26. The method of claim 25 further comprising installing a switch within the bifurcated case to activate and deactivate the indexing markers when the bifurcated case is opened and closed.
